(19) United States
(12) Patent Application Publication
(10) Pub. No.: US 2005/0042285 A1
(43) Pub. Date: Feb. 24, 2005

(54) Stabilized composition comprising a benzimidazole type compound

(75) Inventors: Koji Ukai, Gifu-shi (JP); Masaki Ichikawa, Ibaraki (JP); Takashi Kato, Aichi (JP); Yukiko Sugaya, Ibaraki (JP); Yasuyuki Suzuki, Ibaraki (JP); Shigeru Aoki, Gifu (JP); Akira Kato, Ibaraki (JP); Masao Kawamura, Saitama (JP); Satoshi Fujioka, Aichi (JP)

Correspondence Address: NIXON & VANDERHYE, PC, 1100 N GLEBE ROAD, 8TH FLOOR, ARLINGTON, VA, 22201-4714, US

(73) Assignee: Eisai Co., Ltd., Tokyo (JP)

(21) Appl. No.: 10/938,554

(22) Filed: Sep. 13, 2004

Related U.S. Application Data
(63) Continuation of application No. 09/462,633, filed on Jan. 27, 2000, now abandoned.

(30) Foreign Application Priority Data
Apr. 20, 1998 (JP) 10-109288

(51) Int. Cl. A61K 31/4439; A61K 9/20

(52) U.S. Cl. 424/464; 514/338

(57) ABSTRACT

The present invention provides a chemically stable pharmaceutical preparation of a benzimidazole type compound. That is, the present invention relates to a composition comprising at least one substance selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound or an alkali metal salt thereof.

### FIELD OF THE INVENTION

The present invention relates to pharmaceutical preparations of the solid dosage form for internal use comprising benzimidazole type compounds or alkali metal salts thereof.

### PRIOR ART

A benzimidazole type compound or an alkali metal salt thereof has a strong inhibitory action on the so-called proton pump, and it is widely used as a therapeutic agent for stomach ulcer, duodenal ulcer etc., by inhibiting gastric acid secretion. On the other hand, the benzimidazole type compound is chemically very unstable, so various measures have been invented for pharmaceutical manufacturing thereof. For example, JP-A 62-277322 discloses a process for producing a stabilized pharmaceutical composition comprising a basic inorganic salt of magnesium and/or calcium incorporated into a benzimidazole type compound, and JP-A 62-258320 discloses an oral pharmaceutical preparation prepared by incorporating an alkali compound into the portion of a core containing a benzimidazole type compound, then coating it with fillers for tablets soluble in water or rapidly degradable with water or with a polymeric and water-soluble film-forming compound, and further coating it with an enteric coating.

However, the stability of such pharmaceutical preparations is still insufficient even by the prior art described above, so there is demand for further improvements. That is, the object of the present invention is to further stabilize a pharmaceutical preparation of the solid dosage form for internal use comprising a benzimidazole type compound.

### DISCLOSURE OF THE INVENTION

The present invention relates to a composition comprising at least one selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by the structural formula (formula 1) below or an alkali metal salt thereof.

Formula 1

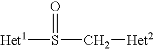

In the formula 1, Het¹ is 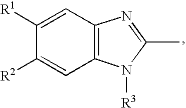
Het² is 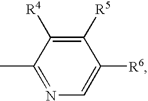
R¹ and R² are the same as or different from each other and are selected from a hydrogen, a methoxy and a difluoromethoxy, R³ is selected from a hydrogen and a sodium, R⁴, R⁵ and R⁶ are the same as or different from each other and are selected from a hydrogen, a methyl, a methoxy, a methoxypropoxy and a trifluoroethoxy.

Further, the present invention relates to a pharmaceutical preparation comprising a core which comprises at least one selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by formula 1 or an alkali metal salt thereof, is coated with an enteric coating.

Further, the present invention relates to a pharmaceutical preparation comprising a core which comprises at least one selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by formula 1 or an alkali metal salt thereof, coated with an intermediate coating and further with an enteric coating.

The present invention further relates to a pharmaceutical preparation comprising a core which comprises at least one selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by formula 1 or an alkali metal salt thereof, coated with an intermediate coating, further with an enteric coating and then with a moisture resistant coating.

The present invention relates to a pharmaceutical composition comprising (A) benzimidazole type compound represented by formula 1 or an alkali metal salt thereof and (B) at least one substance selected from the group consisting of sodium carbonate, potassium carbonate, sodium hydroxide, potassium hydroxide, aminoalkyl methaacrylate copolymer E, arginine aspartate, hydroxypropyl cellulose and crospovidone.

Further, the present invention relates to a pharmaceutical preparation comprising a core consisting of the composition described above and an enteric coating. The pharmaceutical preparation may comprise an intermediate coating, an enteric coating and a moisture resistant coating besides the core.

The moisture resistant coating is effective not only for the benzimidazole type compound but also for a drug whose decomposition is observed to be accelerated both in the presence of water and upon contact with gastric acid. That is, the present invention relates to a pharmaceutical preparation comprising a core coated with an enteric coating and further with a moisture resistant coating, said core comprising a drug incorporated into it and the drug both being accelerated to be decomposed in the presence of water and being chemically unstable in gastric acid.

Further, the present invention relates to a pharmaceutical preparation comprising a core coated with an intermediates coating, further with an enteric coating and then with a moisture resistant coating, said core comprising a drug incorporated into it and the drug both being accelerated to be decomposed in the presence of water and being chemically unstable in gastric acid.

In the present invention, the benzimidazole type compounds or alkali metal salts thereof include e.g. rabeprazole, omeprazole, pantoprazole and lansoprazole, or sodium or potassium salts thereof. The structural formulae of these compounds are shown in formula 3.
Formula 3

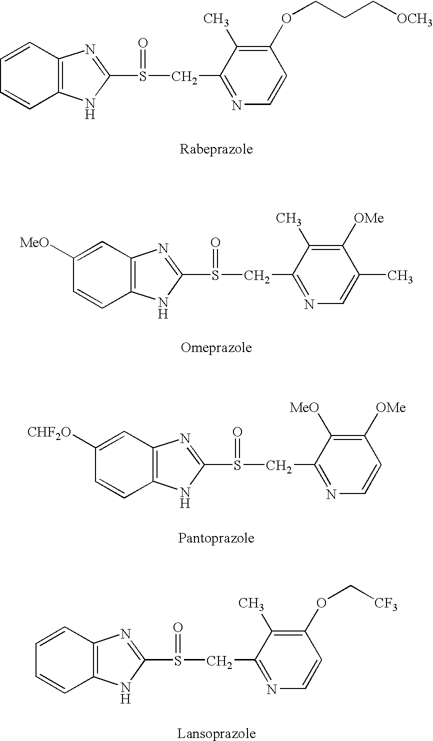

Hereinafter, the benzimidazole type compound or an alkali metal salt thereof is collectively referred to as benzimidazole type compound.

The benzimidazole type compound in the present invention can be produced in a known method. For example, the compound can be produced by any methods disclosed in JP-A 52-62275, JP-A 54-141783, JP-A 1-6270 etc.

Sodium carbonate, potassium carbonate, sodium hydroxide, potassium hydroxide and hydroxypropyl cellulose in the present invention are mentioned in the Japanese Pharmacopoeia, and these are commercially available and easily obtainable. Aminoalkyl methaacrylate copolymer E, which is mentioned in the standards of non-medicines in the Japanese Pharmacopoeia, can be easily obtained. Further, crospovidone is a substance mentioned in the standards of pharmaceutical additives, and its commercial products of various grades with varying particle diameters are easily available, and their particle diameters can be regulated as necessary by a grinding device such as hammer mill.

The blending ratio of the benzimidazole type compound to at least one selected from sodium carbonate, potassium carbonate, sodium hydroxide, potassium hydroxide, aminoalkyl methaacrylate copolymer E, arginine aspartate, hydroxypropyl cellulose and crospovidone is 0.01 to 20 parts by weight, preferably 0.01 to 10 parts by weight, more preferably 0.1 to 10 parts by weight in total, to 1 part by weight of the benzimidazole type compound. In the present invention, sodium carbonate, potassium carbonate, sodium hydroxide, potassium hydroxide, aminoalkyl methaacrylate copolymer E, arginine aspartate, hydroxypropyl cellulose and crospovidone can be used alone or 2 or more of these additives can be used in combination. Among these, it is effective to incorporate sodium hydroxide, potassium hydroxide and/or sodium carbonate into the benzimidazole type compound and it is more effective to incorporate 1) crospovidone and 2) sodium hydroxide, potassium hydroxide and/or sodium carbonate into the benzimidazole type compound. The blending ratio of a combination of these additives is 0.01 to 20 parts by weight to 1 part by weight of the benzimidazole type compound, and preferably the ratio of crospovidone is 0.5 to 5 parts by weight, and the ratio of sodium hydroxide, potassium hydroxide and/or sodium carbonate is 0.01 to 2 parts by weight.

The benzimidazole type compound when decomposed during storage under heating and humid conditions is observed to undergo significant coloring changes in particular. The composition and/or the pharmaceutical preparation of the invention comprising the above-described various additives incorporated into it possesses the particularly outstanding effect of not only improving the stability of the ingredients but also inhibiting the coloring changes.

Conventionally used excipients such as lactose and mannitol can be used to prepare a pharmaceutical preparation by use of the invented composition comprising the benzimidazole type compound and at least one substance selected from sodium carbonate, potassium carbonate, sodium hydroxide, potassium hydroxide, aminoalkyl methaacrylate copolymer E, arginine aspartate, hydroxypropyl cellulose and crospovidone incorporated thereto. Preferably, hydroxypropyl cellulose is used as a binder and crospovidone is used as a disintegrating agent.

It is known that crospovidone used generally as a disintegrating agent, when finely ground, can reduce the disintegrating force and swelling force inherent in the original disintegrating agent. Finely ground crospovidone having small particle diameters is used as a stabilizer for the benzimidazole type compound in the present invention, and it can be added in a larger amount than the amount of a usual disintegrating agent (usually 10% or less). The average particle diameter of crospovidone is several μm to 50 μm, more preferably 4 μm to 50 μm.

Accordingly, the crospovidone used in the composition or in the pharmaceutical preparation according to the present invention is preferably crospovidone having small average particle diameters of several μm to 50 μm, preferably 4 μm to 50 μm. As a matter of course, finely ground crospovidone and usual crospovidone may be used in combination.

The crospovidone, though varying depending on manufacturer and lot number, often contains a slight amount of peroxides as impurities. The benzimidazole type compound is inherently liable to oxidation so that when blended along with crospovidone, it may contain an antioxidant.

The antioxidant includes, but is not limited to, sodium sulfite, sodium pyrosulfite, vitamin E, rongalite, thioglycerol, sodium thiosulfate, ascorbate and acetyl cysteine.

Further, the present invention relates to a pharmaceutical preparation comprising a core which comprises at least one substance selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by formula 1, coated with an enteric coating. In the present invention, the term “core” refers to tablets, granules etc. Further, the present invention encompasses a pharmaceutical preparation comprising a core coated with an enteric coating, said core comprising a benzimidazole type compound and at least one selected from sodium carbonate, potassium carbonate, sodium hydroxide, potassium hydroxide, aminoalkyl methaacrylate copolymer E, arginine aspartate, hydroxypropyl cellulose and crospovidone laminated therein or coated thereon with spherical granules consisting, as seed granules, of refined white sugar, a mixture of white sugar and starch, or crystalline cellulose etc. The benzimidazole type compound is very unstable under acidic conditions, so when administered, the benzimidazole type compound is decomposed immediately in contact with gastric acid in the stomach, to lose its physiological activity. Accordingly, it should be formed as a pharmaceutical preparation not dissolved in the stomach, that is, a pharmaceutical preparation having a benzimidazole type compound-containing core coated with an enteric substance in order to prevent it from being decomposed in the stomach.

Further, the present invention relates to a pharmaceutical preparation comprising a core coated with an intermediate coating and further with an enteric coating, said core comprising at least one substance selected from sodium carbonate, potassium carbonate, sodium hydroxide, potassium hydroxide, aminoalkyl methaacrylate copolymer E, arginine aspartate, hydroxypropyl cellulose and crospovidone incorporated into a benzimidazole type compound represented by formula 1. Since the enteric coating is made generally of an acidic substance, its direct contact with the benzimidazole type compound is not preferable. Accordingly, an inert intermediate coating can be provided between the core comprising a benzimidazole type compound and the enteric coating. The term “inert” refers to a substance not adversely affecting the stability of the benzimidazole type compound. The inert intermediate coating may be made of a water-soluble polymer, a water-soluble or water-disintegrating substance or a water-insoluble substance, and specific examples include hydroxypropyl cellulose, hydroxypropylmethyl cellulose, aminoalkyl methaacrylate copolymer E, lactose, mannitol, starch, crystalline cellulose, ethyl cellulose, vinyl acetate etc. When an intermediate coating made of a water-insoluble substance is applied, water-insoluble fine particles may be mixed in the coating, as disclosed in JP-A 1-290628.

In the present invention, the above-described pharmaceutical preparation coated with an enteric coating may be coated with a moisture resistant coating. The moisture resistant coating is a coating for inhibiting the passage of steam, and it is functionally a coating which in itself inhibits the transmission of steam or a coating which captures steam in the coating to inhibit the inflow of steam into the inside.

The moisture resistant coating possesses the function of defending the preparation against invasion of water into the benzimidazole type compound to improve its stability while preventing the cracking and deformation of tablets originating from the swelling of finely ground crospovidone upon moisture absorption.

The moisture resistant coating may be either a water-soluble coating or a water-insoluble coating, and this coating includes, but is not limited to, a coating consisting of e.g. polyvinyl acetal diethyl aminoacetate, HA Sankyo (a mixture of polyvinyl acetal diethyl aminoacetate, hydroxypropylmethyl cellulose, stearic acid and fumaric acid), polyvinyl alcohol etc., a coating comprising at least one of cellulose derivatives such as hydroxypropyl cellulose, hydroxypropylmethyl cellulose and ethyl cellulose incorporated into it, and/or a sugar coating based on white sugar.

The moisture resistant coating is useful not only for the benzimidazole type compound but also for a pharmaceutical preparation containing a drug having similar chemical properties. That is, its effect is observed to be significant when it is applied onto a pharmaceutical preparation containing a drug whose decomposition is observed to be accelerated both in the presence of water and upon contact with gastric acid.

That is, the present invention relates to a pharmaceutical preparation comprising a core which comprises a drug incorporated into it, the drug both being accelerated to be decomposed in the presence of water and being chemically unstable in gastric acid, coated with an enteric coating and further with a moisture resistant coating. Further, an intermediate coating may be coated between the enteric coating and the moisture resistant coating.

In the present invention, the effect is particularly outstanding where the benzimidazole type compound shown in formula 1 is rabeprazole.

That is, the present invention relates to a composition comprising sodium hydroxide, potassium hydroxide and/or sodium carbonate incorporated preferably into rabeprazole shown in formula 3 or an alkali metal salt thereof.

Further, the present invention relates to a composition comprising 1) crospovidone and 2) sodium hydroxide, potassium hydroxide and/or sodium carbonate incorporated preferably into rabeprazole shown in formula 3 or an alkali metal salt thereof.

As described above, the crospovidone used is preferably finely ground until its average particle diameter is decreased to several μm to 50 μm. Further, an antioxidant may be added to prevent the influence of trace peroxides contained in crospovidone, as described above. Accordingly, an antioxidant may be incorporated into the composition comprising 1) crospovidone and 2) sodium hydroxide, potassium hydroxide and/or sodium carbonate incorporated into rabeprazole or an alkali metal salt thereof.

The present invention relates further to a pharmaceutical preparation comprising a core which comprises 1) crospovidone and 2) sodium hydroxide, potassium hydroxide and/or sodium carbonate incorporated preferably into rabeprazole shown in formula 3 or an alkali metal salt, coated with an enteric coating.

The present invention relates further to a pharmaceutical preparation comprising a core which comprises 1) crospovidone and 2) sodium hydroxide, potassium hydroxide and/or sodium carbonate incorporated preferably into rabeprazole shown in formula 3 or an alkali metal salt, coated with an intermediate coating and further with an enteric coating.

The present invention relates further to a pharmaceutical preparation comprising a core which comprises 1) crospovidone and 2) sodium hydroxide, potassium hydroxide and/or sodium carbonate incorporated preferably into rabeprazole shown in formula 3 or an alkali metal salt, coated with an intermediate coating, further with an enteric coating and then with a moisture resistant coating.

The composition or the pharmaceutical preparation according to the present invention can be produced by any conventionally used processes.

For example, at least one selected from sodium carbonate, potassium carbonate, sodium hydroxide, potassium hydroxide, aminoalkyl methaacrylate copolymer E, arginine aspartate, hydroxypropyl cellulose and crospovidone is incorporated into a benzimidazole type compound or an alkali metal salt thereof, then excipients are added thereto, and the mixture granulated in a dry or wet granulating process, followed by adding a disintegrating agent such as crospovidone as necessary and subsequently tabletting the granules whereby the composition or the pharmaceutical preparation of the invention can be produced. Alternatively, for example, at least one substance selected from sodium carbonate, potassium carbonate, sodium hydroxide, potassium hydroxide, aminoalkyl methaacrylate copolymer E, arginine aspartate, hydroxypropyl cellulose and crospovidone is incorporated at high density into a benzimidazole type compound or an alkali metal salt to prepare benzimidazole-containing granules, while placebo granules not containing the benzimidazole type compound are separately prepared, and then both the granules are mixed followed by adding a disintegrating agent such as crospovidone as necessary and subsequently tabletting the granules. As a matter of course, these processes are non-limiting examples.

In a concrete example, e.g. 100 g sodium rabeprazole as the benzimidazole type compound, 30 g sodium carbonate and 130 g mannitol are mixed, and hydroxypropyl cellulose dissolved in ethanol is gradually added to the mixture under stirring, followed by granulation, drying and screening through a 24-mesh screen. 30 g crospovidone and 2 g calcium stearate are added thereto, mixed and tabletted whereby tablets each weighing 135 mg can be obtained.

These tablets are sprayed by using a fluidized-bed granulator with a solution of hydroxypropyl cellulose in ethanol and further with a solution of hydroxypropylmethyl cellulose phthalate or an enteric methaacrylate copolymer in water/ethanol whereby enteric tablets provided with an intermediate coating can be produced.

According to the present invention, it is possible to stabilize the very unstable benzimidazole type compound. Examples of this effect are shown below.

### EXPERIMENTAL EXAMPLES

50 mg sodium rabeprazole and 450 mg additives shown in the table below were mixed in a mortar.

The mixture was introduced into a transparent glass vial and stored in a cold place or at 60° C. or 40 C under 75% relative humidity for 1 week and their content was determined by high performance liquid chromatography. Assuming that the content of the sample stored in the cold place is 100%, the degrees of the residual content under the respective conditions are shown in Tables 1 through 3. Further, their coloring changes were visually evaluated. The sodium rabeprazole used was amorphous in Table land crystalline in Tables 2 and 3. In Table 1, low-substituted hydroxypropyl cellulose (expressed as L-HPC) used as a disintegrating agent in addition to amorphous sodium rabeprazole was blended in the control; in Table 2, a sample further incorporating aluminum hydroxide (expressed as Al(OH)₃ in the table) i.e. an alkaline inorganic salt used as an antacid agent was used; and in Table 3, a sample incorporating polyvinyl pyrrolidone (expressed as PVP in the table) was used as a binder.

TABLE 1
Compatibility Test of Sodium Rabeprazole

| | | 60° C. | 40° C.-75% RH |
|---|---|---|---|
| Control | sodium rabeprazole | 99.1 | 93.9 |
| | (amorphous) | | |
| | sodium rabeprazole + | 80.4 | 73.3 |
| | L-HPC | | |
| The present | sodium rabeprazole + | 98.1 | 90.4 |
| application | crospovidone | | |

Unit: %

TABLE 2
Compatibility Test of (crystalline) Sodium Rabeprazole

| | | 60° C. | 40° C.-75% RH |
|---|---|---|---|
| Control | sodium rabeprazole | 99.8 | 91.8 |
| | (crystalline) | | |
| | sodium rabeprazole + L-HPC | 62.2 | 75.0 |
| | sodium rabeprazole + | 36.9 | 26.2 |
| | Al(OH)₃ | | |
| The present | sodium rabeprazole + | 93.3 | 89.5 |
| | crospovidone | | |
| application | sodium rabeprazole + Na₂CO₃ | 99.1 | 90.3 |
| | sodium rabeprazole + | 97.5 | 90.7 |
| | Arg.Asp | | |

Unit: %

TABLE 3
Compatibility Test of (Crystalline) Sodium Rabeprazole

| | | 60° C. | 40° C.-75% RH |
|---|---|---|---|
| Control | sodium rabeprazole | 97.3 | 86.9 |
| | (crystalline) | | |
| | sodium rabeprazole + PVP | 89.5 | 67.7 |
| The present | sodium rabeprazole + | 92.0 | 86.9 |
| application | hydroxypropyl cellulose | | |
| | sodium rabeprazole + Na₂CO₃ | 93.0 | 82.8 |
| | sodium rabeprazole + NaOH | 91.6 | 98.8 |
| | sodium rabeprazole + KOH | 92.6 | 96.8 |
| | sodium rabeprazole + | 102.4 | 86.0 |
| | Eudragit E | | |
| | sodium rabeprazole + K₂CO₃ | 104.5 | 81.3 |

Unit: %

Any coloring changes of the blended samples according to the present invention were lower than those of the controls. Further, it is evident from the results of content stability in Tables 1 through 3 that the ingredients used in the present invention, that is, sodium carbonate (expressed as Na₂CO₃ in the table), sodium carbonate (expressed as K₂CO₃ in the table) sodium hydroxide (expressed as NaOH in the table), potassium hydroxide (expressed as KOH), aminoalkyl methaacrylate copolymer E (expressed as Eudragit E®), arginine aspartate (expressed as Arg Asp in the table), hydroxypropyl cellulose and crospovidone stabilize the benzimidazole type compound.

Effect of Sodium Carbonate in Tablets

Tablets containing different amounts of sodium carbonate, obtained in Examples 4 to 9 shown below, were stored at 40° C. under 75% relative humidity for 1 week, and the contents of sodium rabeprazole in the tablets as determined by high performance liquid chromatography were shown in Table 4.

TABLE 4
Stability Evaluation of Tablet Formulations by Wet Granulation

| Formulation | Ex. 4 | Ex. 5 | Ex. 6 | Ex. 7 | Ex. 8 | Ex. 9 |
|---|---|---|---|---|---|---|
| (1 week) | | | | | | |
| cold place | 99.4 | 99.0 | 98.7 | 99.4 | 99.5 | 98.9 |
| 40° C.-75% RH | 83.8 | 85.7 | 85.1 | 92.5 | 92.8 | 95.5 |
| (1 month) | | | | | | |
| cold place | 99.7 | 99.7 | 99.7 | 99.7 | 99.7 | 99.6 |
| 25° C.-75% RH | 97.8 | 98.5 | 98.3 | 99.2 | 99.3 | 99.3 |

Unit: %

Because the stability of the content of sodium rabeprazole in the tablets is improved depending on the amount of sodium carbonate added, the effect of sodium carbonate added in the present invention is evident.

Effect of Crospovidone in Tablets

Tablets containing different amounts of crospovidone powder, obtained in Examples 10 to 12 shown below, were stored at 40° C. under 75% relative humidity for 1 week, and the contents of sodium rabeprazole in the tablets as determined by high performance liquid chromatography were shown in Table 5. The tablets were subject to less coloring change as the amount of the crospovidone powder added was increased.

TABLE 5
Stability of Crospovidone-Added Tablets by Wet Granulation

| Formulation | Ex. 10 | Ex. 11 | Ex. 12 |
|---|---|---|---|
| (1 week) | | | |
| cold place | 99.7 | 99.7 | 99.7 |
| 40° C.-75% RH | 97.8 | 98.5 | 98.3 |
| (1 month) | | | |
| cold place | 99.4 | 99.0 | 98.7 |
| 40° C.-75% RH | 83.8 | 85.7 | 85.1 |

Unit: %

It is evident that the stability of the benzimidazole type compound is improved by adding crospovidone.

### Effect of Finely Ground Crospovidone in Tablets

Tablets containing crospovidone having a different average particle diameter, obtained in Examples 16 to 18 shown below, were stored in a cold place or at 25° C. under 75% relative humidity for 1 month and then evaluated for their thickness to evaluate the ratio of swelling of the tablets stored at 25° C. under 75% relative humidity to swelling of the tablets stored in the cold place. The results were that the ratios of swelling of the tablets containing crospovidone having average particle diameters of 51 μm, 12 μm and 6 μm were 1.61, 1.48 and 1.43, respectively.

As crospovidone is made fine powder having a small average particle diameter, the ratio of the swelling of the tablets is decreased. Therefore, the cracking or deformation resulting from the swelling of the tablets is reduced. Accordingly, it is evident that the particle size reduction of crospovidone contributes to improvement of stability of tablets.

Effect of a Moisture Resistant Coating Applied onto Tablets Coated with an Enteric Coating

Tablets coated with an enteric coating and tablets coated with both an enteric coating and a moisture resistant coating, obtained in Examples 19 to 20 shown below, were stored at 25° C. under 75% relative humidity for 1 week, and the content of a rabeprazole analogue in the tablets was determined by high performance liquid chromatography. The results indicated that the contents of the rabeprazole analogue in the tablets coated with an enteric coating and the tablets coated with both an enteric coating and a moisture resistant coating were 2.38% and 2.23%, respectively.

It is evident that the tablets coated with both an enteric coating and a moisture resistant coating possess stability equal to or higher than that of the tablets coated with an enteric coating.

Placebo tablets obtained in Examples 21 to 23 shown below were stored in a cold place or at 40° C. under 75% relative humidity for 1 week and then evaluated for their thickness to evaluate the ratio of swelling of the tablets stored at 40° C. under 75% relative humidity to swelling of the tablets stored in the cold place. The results indicated that the ratios of swelling of the tablets coated with an enteric coating, tablets prepared by coating said enteric coating-coated tablets with a moisture resistant coating, and tablets prepared by coating said enteric coating-coated tablets with a moisture resistant coating consisting of HA (Sankyo) (i.e., a mixture of polyvinyl acetal diethyl aminoacetate, hydroxypropylmethyl cellulose, macrogol and talc) were 1.15, 1.03 and 1.12, respectively.

Since the degree of swelling of the tablets coated with both an enteric coating and a moisture resistant coating is smaller during storage than that of the tablets coated with an enteric coating only, it is evident that the stability in shape of the tablets is improved.

Effect of an Antioxidant Added to the Portion of a Core Containing the Benzimidazole Type Compound

Tablets containing a different amount of a peroxide, obtained in Examples 24 to 26 shown below, were measured for the content of a sodium rabeprazole analogue by high performance liquid chromatography. The results indicate that the amounts of the initial rabeprazole analogue in the tablets incorporating crospovidone containing 18 ppm, 190 ppm and 310 ppm peroxide were 0.65%, 0.88% and 1.13% respectively, indicating that as the amount of the peroxide in crospovidone is increased, the decomposition of sodium rabeprazole is promoted to increase the amount of the analogue.

Further, 1 g crospovidone containing 201 ppm peroxide was accurately taken, and sodium sulfite (amounts: 4 levels i.e. no addition, 0.02%, 0.05% and 0.10%) was added thereto and mixed well, and the amount of the peroxide in the mixture was determined according to a test method described in the Japanese Pharmacopoeia. The results indicated that the amounts of the peroxide in the compositions wherein the amounts of sodium sulfite added were none, 0.02%, 0.05% and 0.10%, were 201 ppm, 184 ppm, 108 ppm, and 0 ppm respectively, indicating that as the amount of sodium sulfite added was increased, the amount of the peroxide was reduced.

From the foregoing, it is evident that the stability of the benzimidazole type compound in a pharmaceutical preparation is improved by adding the antioxidant to the portion of cores in tablets containing the benzimidazole type compound and crospovidone.

### EXAMPLES

Hereinafter, the present invention is described more in detail by reference to Examples, which however are not intended to limit the present invention.

### Example 1

10 g sodium carbonate and 100 g mannitol were added to and mixed with 10 g sodium rabeprazole, and 2.5 g hydroxypropyl cellulose dissolved in ethanol was gradually added to the mixture under stirring to make granules which were dried and screened followed by adding calcium stearate and tabletting to give tablets each weighing 120 mg containing 10 mg sodium rabeprazole.

### Example 2

The tablets obtained in Example 1 were sprayed by using a fluidized-bed granulator with a solution of 10 g hydroxypropylmethyl cellulose phthalate dissolved in a mixed solvent of water and ethanol (2:8), to produce enteric tablets.

### Example 3

The tablets obtained in Example 1 were sprayed by using a fluidized-bed granulator with a solution of hydroxypropylmethyl cellulose in ethanol, to produce enteric tablets in the same manner as in Example 2.

### Examples 4 to 9

0 to 10 g sodium carbonate and 15 to 90 g mannitol were added to and mixed with 10 g sodium rabeprazole, and 0.7 to 2 g hydroxypropyl cellulose dissolved in ethanol was gradually added to the mixture to make granules under stirring in a wet granulation process, thus preparing the active granules. Separately, 2 g hydroxypropyl cellulose dissolved in ethanol was gradually added to 100 g mannitol to produce granules under stirring in a wet process to prepare placebo granules. Then, the main-drug granules were mixed with the placebo granules, and 5% crospovidone and a slight amount of magnesium stearate were added thereto in a powdery form and tabletted to give tablets each weighing 100.5 mg containing 10 mg sodium rabeprazole. Each formulation is shown in Table 6.

TABLE 6
Tablet Formation by Wet Granuration

| Formulation | | Ex. 4 | Ex. 5 | Ex. 6 | Ex. 7 | Ex. 8 | Ex. 9 |
|---|---|---|---|---|---|---|---|
| Active | sodium | 10.0 | 10.0 | 10.0 | 10.0 | 10.0 | 10.0 |
| granule | rabeprazole | | | | | | |
| | anhydrous | — | — | — | 5.0 | 5.0 | 10.0 |
| | sodium | | | | | | |
| | carbonate | | | | | | |
| | mannitol | 82.0 | 30.0 | 20.0 | 25.0 | 15.0 | 20.0 |
| | hydroxypropyl | 2.0 | 1.0 | 0.7 | 1.0 | 0.7 | 1.0 |
| | cellulose | | | | | | |
| | (sub-total) | 94.0 | 41.0 | 30.7 | 41.0 | 30.7 | 41.0 |
| Placebo | mannitol | — | 52.0 | 62.1 | 52.0 | 62.1 | 52.0 |
| granule | hydroxypropyl | — | 1.0 | 1.2 | 1.0 | 1.2 | 1.0 |
| | cellulose | | | | | | |
| | (sub-total) | 0.0 | 53.0 | 63.3 | 53.0 | 63.3 | 53.0 |
| Powder | crospovidone | 5.0 | 5.0 | 5.0 | 5.0 | 5.0 | 5.0 |
| added | magnesium | 1.5 | 1.5 | 1.5 | 1.5 | 1.5 | 1.5 |
| | stearate | | | | | | |
| | (sub-total) | 6.5 | 6.5 | 6.5 | 6.5 | 6.5 | 6.5 |
| total | | 100.5 | 100.5 | 100.5 | 100.5 | 100.5 | 100.5 |

Unit: mg

### Examples 10 to 12

Tablets were obtained in the same manner as in Examples 4 to 9 except that the amounts of crospovidone powder added were 3 levels, that is, 0, 2.5, and 5%. Each formulation is shown in Table 7.

TABLE 7
Formulation of Crospovidone-Added Tablets by Wet Granulation

| Formulation | | Ex. 10 | Ex. 11 | Ex. 12 |
|---|---|---|---|---|
| Active | sodium rabeprazole | 10.0 | 10.0 | 10.0 |
| granule | (crystalline) | | | |
| | anhydrous | 5.0 | 5.0 | 5.0 |
| | sodium carbonate | | | |
| | mannitol | 25.0 | 25.0 | 25.0 |
| | hydroxypropyl cellulose | 1.0 | 1.0 | 1.0 |
| | (sub-total) | 41.0 | 41.0 | 41.0 |
| Placebo | mannitol | 56.9 | 54.4 | 52.0 |
| granule | hydroxypropyl cellulose | 1.1 | 1.1 | 1.0 |
| | (sub-total) | 58.0 | 55.5 | 53.0 |
| Powder | crospovidone | — | 2.5 | 5.0 |
| added | magnesium stearate | 1.5 | 1.5 | 1.5 |
| | (sub-total) | 1.5 | 4.0 | 6.5 |

| | | | |
|---|---|---|---|
| total | 100.5 | 100.5 | 100.5 |

Unit: mg

### Examples 13 to 14

According to the 2 formulations shown in Table 8, 0 to 50 g sodium carbonate, 79.3 to 84.3 g mannitol, 4.2 g crospovidone and 1.5 g magnesium stearate were added to 10 mg sodium rabeprazole, mixed well, and directly tabletted to give tablets each weighing 100 mg containing 10 mg sodium rabeprazole.

TABLE 8
Tablet Formulation by Direct Tabletting

| Formulation | Ex. 13 | Ex. 14 |
|---|---|---|
| sodium rabeprazole | 10.0 | 10.0 |
| (crystalline) | | |
| anhydrous | — | 5.0 |
| sodium carbonate | | |
| mannitol | 84.3 | 79.3 |
| crospovidone | 4.2 | 4.2 |
| magnesium stearate | 1.5 | 1.5 |
| total | 100.0 | 100.0 |

Unit: mg

### Example 15

50 g sodium carbonate and 2 g magnesium stearate were added to 100 g sodium rabeprazole, mixed well to make granules under dry compression granulation process, to prepare main-drug granules. Separately, 76.3 g mannitol was added to and mixed well with 4.2 g crospovidone, and 2.3 g hydroxypropyl cellulose dissolved in ethanol was gradually added thereto to make granules under stirring in a wet process to prepare placebo granules. Then, the main-drug granules were mixed with the placebo granules, and a slight amount of magnesium stearate was added thereto in a powdery form and tabletted to give tablets each weighing 100 mg containing 10 mg sodium rabeprazole as shown in Table 9.

TABLE 9
Tablet Formulation by Dry Granulation

| Formulation | | Ex. 15 |
|---|---|---|
| Active | sodium rabeprazole | 10.0 |
| granule | (crystalline) | |
| | anhydrous | 5.0 |
| | sodium carbonate | |
| | magnesium stearate | 0.2 |
| | (sub-total) | 15.2 |
| Placebo | mannitol | 76.8 |
| granule | crospovidone | 4.2 |
| | hyroxypropyl cellulose | 2.3 |
| | (sub-total) | 83.3 |
| Powder | magnesium stearate | 1.5 |
| added | | |

| | |
|---|---|
| total | 100.0 |

Unit: mg

### Examples 16 to 18

527 g crospovidone having a different average particle diameter and 20 g hydroxypropyl cellulose were mixed with 100 g sodium rabeprazole, and 3 g magnesium stearate was added thereto in a powdery form, followed by tabletting to give tablets each weighing 65 mg containing 10 mg sodium rabeprazole as shown in Table 10. Crospovidone used is a product of BASF Ltd., and its average diameter is 51 μm for Colidone CL™, 12 μm for Colidone CLM™ and 6 μm for a hammer mill-ground product of Colidone CLM™.

TABLE 10
Formulations Containing Crospovidone having
Different Particle Diameters

| Formulation | Ex. 16 | Ex. 17 | Ex. 18 |
|---|---|---|---|
| sodium rabeprazole | 10.0 | 10.0 | 10.0 |
| crospovidone (colidone CL) | 52.7 | — | — |
| crospovidone (colidone CLM) | — | 52.7 | — |
| crospovidone | — | — | 52.7 |
| (ground product of colidone CLM) | | | |
| hydroxypropyl cellulose | 2.0 | 2.0 | 2.0 |
| magnesium stearate | 0.3 | 0.3 | 0.3 |
| (sub-total) | 65.0 | 65.0 | 65.0 |

Unit: mg
Note:
Average diameters
Crospovidone (Colidone CL): 51 μm
Crospovidone (Colidone CLM): 12 μm
Crospovidone (ground product of Colidone CLM): 6 μm

### Examples 19 to 20

The portion of a core containing sodium rabeprazole was granulated with ethanol and coated with a water-insoluble intermediate coating containing ethyl cellulose, crospovidone and magnesium stearate. Further, the resulting granules were coated with a coating to give tablets coated with an enteric coating or with both an enteric coating and a moisture resistant coating. The formulation is shown in Table 11.

TABLE 11
Formulation of a Pharmaceutical Preparation Having an Enteric
Coating and a Moisture Resistant Coating Applied Thereon

| Formulation | | Ex. 19 | Ex. 20 |
|---|---|---|---|
| Core | sodium rabeprazole | 10.0 | 10.0 |
| | mannitol | 36.2 | 36.2 |
| | crospovidone | 15.6 | 15.6 |
| | sodium hydroxide | 0.1 | 0.1 |
| | anhydrous sodium carbonate | 5.0 | 5.0 |
| | hydroxypropyl cellulose | 2.0 | 2.0 |
| | magnesium stearate | 1.1 | 1.1 |
| | (sub-total) | 70.0 | 70.0 |
| Intermediate | ethyl cellulose | 0.5 | 0.5 |
| coating | crospovidone | 1.0 | 1.0 |
| | magnesium stearate | 0.1 | 0.1 |
| | (sub-total) | 1.6 | 1.6 |
| Enteric | hydroxypropyl cellulose | | |
| coating | cellulose phthalate | 8.0 | 8.0 |
| | monoglyceride | 0.8 | 0.8 |
| | talc | 0.75 | 0.75 |
| | titanium oxide | 0.4 | 0.4 |
| | yellow iron oxide | 0.05 | 0.05 |
| | (sub-total) | 10.0 | 10.0 |
| Moisture | hydroxypropylmethyl | — | 3.0 |
| resistant | cellulose | | |
| coating | macrogol | — | 0.6 |
| | talc | — | 1.4 |
| | (sub-total) | | 5.0 |

| | | |
|---|---|---|
| total | 81.6 | 86.6 |

Unit: mg

### Examples 21 to 23

As placebo tablets not containing the benzimidazole type compound, tablets having a water-soluble intermediate layer of hydroxypropyl cellulose applied onto the portion of cores therein were prepared. The tablets were coated further with an enteric coating to prepare tablets coated with an enteric coating, and further the enteric coating-coated tablets were sprayed with white sugar or HA (Sankyo) to prepare tablets coated with a moisture resistant coating. The formulation is shown in Table 12.

TABLE 12
Placebo Formulation

| Formulation | | Ex. 21 | Ex. 22 | Ex. 23 |
|---|---|---|---|---|
| Core | mannitol | 31.8 | 31.8 | 31.8 |
| | crospovidone (colidone CLM) | 27.7 | 27.7 | 27.7 |
| | hydroxypropyl cellulose | 5.0 | 5.0 | 5.0 |
| | magnesium stearate | 0.5 | 0.5 | 0.5 |
| | (sub-total) | 65.0 | 65.0 | 65.0 |
| Intermediate | hydroxypropyl cellulose | 3.0 | 3.0 | 3.0 |
| coating | | | | |
| Enteric | hydroxypropylmethyl | 8.0 | 8.0 | 8.0 |
| coating | cellulose phthalate | | | |
| | monoglyceride | 0.8 | 0.8 | 0.8 |
| | talc | 0.75 | 0.75 | 0.75 |
| | titanium oxide | 0.4 | 0.4 | 0.4 |
| | yellow iron oxide | 0.05 | 0.05 | 0.05 |
| | (sub-total) | 10.0 | 10.0 | 10.0 |
| Moisture | white sugar | — | 10.0 | — |
| resistant | HA (Sankyo)* | — | — | 10.0 |
| coating | | | | |

| | | | |
|---|---|---|---|
| total | 78.0 | 88.0 | 88.0 |

Unit: mg
Note:
HA (Sankyo)* A mixture of polyvinyl acetal diethyl aminoacetate, hydroxypropylmethyl cellulose, Macrogol and talc.

### Examples 24 to 26

Tablets containing crospovidone with different contents of sodium rabeprazole and a peroxide, sodium hydroxide and sodium carbonate were obtained by granulation in a wet process, according to the formulation in Table 13.

TABLE 13
Formulation Containing Crospovidone with
Different Contents of Peroxide

| Formulation | Ex. 24 | Ex. 25 | Ex. 26 |
|---|---|---|---|
| sodium rabeprazole | 10.0 | 10.0 | 10.0 |
| mannitol | 36.9 | 36.9 | 36.9 |
| crospovidone (INF-10)*1 | 14.0 | — | — |
| crospovidone (INF-10)*2 | — | 14.0 | — |
| crospovidone (colidone CLM)*3 | — | — | 14.0 |
| crospovidone (colidone CL) | 14.0 | 14.0 | 14.0 |
| sodium hydroxide | 0.5 | 0.5 | 0.5 |
| anhydrous sodium carbonate | 2.5 | 2.5 | 2.5 |
| hydroxypropyl cellulose | 2.0 | 2.0 | 2.0 |
| magnesium stearate | 1.1 | 1.1 | 1.1 |
| (total) | 70.0 | 70.0 | 70.0 |

Unit: mg
Note:
Crospovidone (INF-10)*1: (peroxide content: 18 ppm)
Crospovidone (INF-10)*2: (peroxide content: 190 ppm)
Crospovidone (Colidone CLM)*3: (peroxide content: 310 ppm)

### Example 27

43.5 g finely ground crospovidone and 6 g hydroxypropyl cellulose were added to 30 g sodium rabeprazole, mixed well, and then a solution of sodium hydroxide in ethanol (solution of 1.5 g sodium hydroxide dissolved in ethanol) was gradually added to the mixture under stirring to make granules, followed by drying and subsequent regulation of the size of granules in a small type speed mill. 3% crospovidone and 1.6% magnesium stearate were added to the regulated granules, mixed and tabletted into tablets each weighing 70 mg containing 10 mg sodium rabeprazole.

### Example 28

The tablets obtained in Example 27 were coated by using a fluidized-layer granulator with a hydrous ethanol solution containing hydroxypropyl cellulose and a slight amount of magnesium stearate, to give tablets having 2 mg intermediate coating laminated thereon. Then, the tablets coated with the intermediate coating were sprayed by using a fluidized-layer granulator with a hydrous ethanol solution containing hydroxypropyl cellulose phthalate, monoglyceride, talc and titanium oxide, to give enteric tablets coated with 10 mg enteric coating.

### Example 29

The enteric tablets obtained in Example 28 were sprayed by using a fluidized-layer granulator with purified water containing hydroxypropylmethyl cellulose, Macrogol 6000™ and talc to give tablets coated with 5 mg moisture resistant coating.

1-15. (Canceled).

16. A composition comprising (A) rabeprazole or an alkali metal salt thereof and (B) sodium hydroxide, wherein the composition comprises 1 part by weight of (A) and 0.01 to 2 parts by weight of (B).

17. A composition comprising (A) rabeprazole or an alkali metal salt thereof, (B) crospovidone and sodium hydroxide, wherein the composition comprises 1 part by weight of (A), 0.01 to 2 parts by weight of sodium hydroxide and 0.5 to 5 parts by weight of crospovidone.

18. A pharmaceutical preparation comprising a core consisting of the composition as claimed in claim 16 or 17 and an enteric coating thereon.

19. A pharmaceutical preparation comprising a core consisting of the composition as claimed in claim 16 or 17, an intermediate coating and an enteric coating.

20. A pharmaceutical preparation comprising a core consisting of the composition as claimed in claim 16 or 17, an intermediate coating, an enteric coating and a moisture resistant coating.

21. The pharmaceutical composition of claim 16 or 17 wherein components (A) and (B) are uniformly mixed with each other.

22. The pharmaceutical composition of claim 16 or 17 wherein (A) is mixed with a portion of (B) to form granules, then a further amount of (B) is added to the granules and mixed, then the mixture is formed into a tablet.

23. The pharmaceutical composition of claim 16 or 17 wherein (A) is mixed with a portion of (B) and formed into a core, then a further amount of (B) is coated as a layer onto the core.

24. A pharmaceutical preparation comprising a core consisting of the composition as claimed in claim 17 and an enteric coating.

25. A pharmaceutical preparation comprising a core consisting of the composition as claimed in claim 17, an intermediate coating and an enteric coating.

26. A pharmaceutical preparation comprising a core consisting of the composition as claimed in claim 17, an intermediate coating, an enteric coating and a moisture-resistant coating.

27. The composition according to claim 17, which further comprises an antioxidant.

28. The pharmaceutical preparation according to any one of claims 24, 25 or 26, wherein the core further comprises an antioxidant.

29. A pharmaceutical preparation comprising a core which comprises the composition of claim 16 or 17 coated with an enteric coating and further with a moisture-resistant coating.

30. A pharmaceutical preparation comprising a core which comprises the composition of claim 16 or 17 coated with an intermediate coating, further with an enteric coating and then with a moisture-resistant coating.

31. A composition comprising (A) rabeprazole or an alkali metal salt thereof and (B) sodium hydroxide, wherein the composition comprises 1 part by weight of (A) and 0.01 to 0.5 parts by weight of (B).

32. A composition comprising (A) rabeprazole or an alkali metal salt thereof, (B) crospovidone and sodium hydroxide, wherein the composition comprises 1 part by weight of (A), 0.01 to 0.5 parts by weight of sodium hydroxide and 0.5 to 5 parts by weight of crospovidone.

33. The composition according to claim 31 or 32 which comprises 1 part by weight of (A) and 0.05 parts by weight of (B).
